(19) United States
(12) Patent Application Publication
(10) Pub. No.: US 2025/0191715 A1
(43) Pub. Date: Jun. 12, 2025

(54) MACHINE LEARNING ADVERSE DRUG REACTION PREDICTION

(71) Applicant: DANIKA GUPTA, Saratoga, CA (US)

(72) Inventor: DANIKA GUPTA, Saratoga, CA (US)

(21) Appl. No.: 18/533,402

(22) Filed: Dec. 8, 2023

(51) Int. Cl. G16H 20/10; G16H 70/40

(52) U.S. Cl. CPC G16H 20/10; G16H 70/40

(57) ABSTRACT

Apparatuses, systems, methods, and computer program products are disclosed for machine learning adverse drug reaction prediction. A method includes processing one or more of chemical structure information, side effect relationship information, target information, and/or indication information for a medication candidate using one or more machine learning models. A method includes generating a list of multiple predicted side effects for a medication candidate based on machine learning processing. A method includes displaying, to a user, a generated list of multiple predicted side effects for a medication candidate on an electronic display screen for a hardware computing device.

### FIELD

This invention relates to adverse drug reactions and more particularly relates to adverse drug reaction prediction using machine learning.

### BACKGROUND

Adverse drug reactions are a leading cause of death and can be difficult to detect during drug development and to track afterward. In addition to the human cost of adverse drug reactions and other side effects, they cost the public and the healthcare system billions of dollars annually.

### SUMMARY

Apparatuses are presented for machine learning adverse drug reaction prediction. In one embodiment, an apparatus includes a processor and a memory storing computer program code executable by the processor to perform operations. An operation, in some embodiments, includes processing one or more of chemical structure information, side effect relationship information, target information, and/or indication information for a medication candidate using one or more machine learning models. An operation, in a further embodiment, includes generating a list of multiple predicted side effects for a medication candidate based on processing using one or more machine learning models. An operation, in certain embodiments, includes displaying, to a user, a generated list of multiple predicted side effects for a medication candidate on an electronic display screen for a hardware computing device.

Computer program products comprising a non-transitory computer readable storage medium are presented. In certain embodiments, a computer readable storage medium stores computer program code executable to perform operations for machine learning adverse drug reaction prediction. In some embodiments, an operation includes processing one or more of chemical structure information, side effect relationship information, target information, and/or indication information for a medication candidate using one or more machine learning models. An operation, in certain embodiments, includes generating a list of multiple predicted side effects for a medication candidate based on processing using one or more machine learning models. In a further embodiment, an operation includes displaying, to a user, a generated list of multiple predicted side effects for a medication candidate on an electronic display screen for a hardware computing device.

Methods are presented for machine learning adverse drug reaction prediction. In one embodiment, a method includes processing one or more of chemical structure information, side effect relationship information, target information, and/or indication information for a medication candidate using one or more machine learning models. A method, in some embodiments, includes generating a list of multiple predicted side effects for a medication candidate based on processing using one or more machine learning models. In certain embodiments, a method includes displaying, to a user, a generated list of multiple predicted side effects for a medication candidate on an electronic display screen for a hardware computing device.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system for machine learning adverse drug reaction prediction;

FIG. 2 is a schematic block diagram illustrating one embodiment of an adverse drug reaction module;

FIG. 3 is a schematic block diagram illustrating a further embodiment of a system for machine learning adverse drug reaction prediction;

FIG. 4 is a schematic flowchart diagram illustrating one embodiment of a method for machine learning adverse drug reaction prediction; and

FIG. 5 is a schematic flowchart diagram illustrating a further embodiment of a method for machine learning adverse drug reaction prediction.

### DETAILED DESCRIPTION

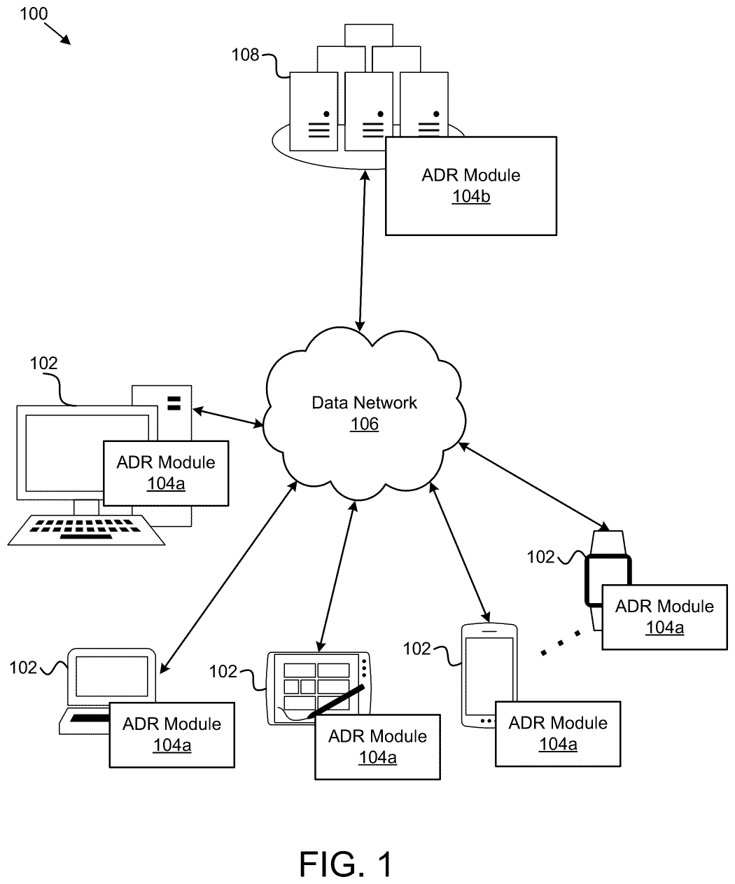

FIG. 1 depicts one embodiment of a system 100 for machine learning adverse drug reaction prediction. In one embodiment, the system 100 includes one or more hardware computing devices 102, one or more adverse drug reaction modules 104 (e.g., one or more adverse drug reaction modules 104a disposed on the one or more hardware computing devices 102, one or more backend adverse drug reaction modules 104b, or the like), one or more data networks 106 or other communication channels, and/or one or more backend server devices 108. In certain embodiments, even though a specific number of hardware computing devices 102, adverse drug reaction modules 104, data networks 106, and/or backend server devices 108 are depicted in FIG. 1, one of skill in the art will recognize, in light of this disclosure, that any number of hardware computing devices 102, adverse drug reaction modules 104, data networks 106, and/or backend server devices 108 may be included in the system 100 for machine learning adverse drug reaction prediction.

In general, an adverse drug reaction module 104, in various embodiments, is configured to receive and/or collect information for a medication candidate (e.g., chemical structure information, side effect relationship information, target information, indication information, a user's medical history, and/or other information associated with a medication candidate and/or a user), to process the received information using one or more machine learning models, to generate a list of multiple predicted side effects for the medication candidate based on the processing, and to display and/or otherwise communicate the predicted side effects to a user (e.g., on an electronic display screen of a hardware computing device 102, or the like).

For example, in certain embodiments, an adverse drug reaction module 104 may employ a hybrid approach to forecasting drug/medication side effects, where molecular or other chemical structure featurization techniques may be combined with knowledge graph embeddings or other machine learning models to create a deep learning model that can combine both chemical structure information and other contextual information for a medication candidate (e.g., regarding intended use and relationship to morbidities, such as side effect relationship information, target information, indication information, or the like).

An adverse drug reaction module 104 may help doctors, hospitals, patients, and/or other users avoid one or more problems that come with adverse drug reactions (ADRs) and other side effects and may help patients expect and/or manage side effects better via a holistic approach that combines chemical structure information and human body interactions and/or other medication candidate information (e.g., side effect relationship information, target information, indication information, or the like). As a drug or other medication is given to patients and more information is gathered about how patients react, in some embodiments an adverse drug reaction module 104 may learn from this newly added information and predict side effects even better over time.

By combining multiple modalities (e.g., chemical structure information, side effect relationship information, target information, indication information, or the like), in certain embodiments, an adverse drug reaction module 104 may create a more holistic approach to ADR and/or side effect forecasting for medication candidates. For example, an adverse drug reaction module 104 may process information for a medication candidate using one or more machine learning models, such as one or more deep learning models, multitask classifiers (e.g., a robust multitask classifier, or the like), graph convolutional neural networks (e.g., for multi-label classification or the like), knowledge graph (KG) embedding models (e.g., with nodes for chemical structure information, side effect relationship information, target information, indication information, multiple potential/predicted side effects, or the like), link completion models, K nearest neighbors models, random forest models, or the like to leverage both drug target information (e.g., a protein target, a bacterial target, a viral target, a fungal target, a cellular target, or the like) and indication information (e.g., a reason to use a medication candidate, label indications, off-label indications, symptoms, illnesses, conditions, and/or other indications) to predict side effects.

In some embodiments, the processing of multiple types of information (e.g., chemical structure information, side effect relationship information, target information, indication information, medical history information, or the like) using one or more machine learning models may improve side effect prediction accuracy. For example, in certain embodiments, predicting side effects based solely on drug chemical structure information may be challenging and/or less accurate, and an adverse drug reaction module 104 may also process related side effect information, target information, and/or indication information (e.g., depending on which information is available for a given new medication candidate, or the like) to improves prediction accuracy.

An adverse drug reaction module 104 may forecast one or more side effects of a new drug or other medication candidate by combining chemical structure information, side effect relationship information, target information, and/or indication information using one or more machine learning models. For example, in some embodiments, an adverse drug reaction module 104 may use a knowledge graph and combine it with one or more deep learning models that can learn the chemical structure of a medication candidate. An adverse drug reaction module 104, in further embodiments, may add additional information such as dosage for a medication candidate to a machine learning processing pipeline, to determine one or more side effects. As drugs or other medications are given to patients and more information is gathered about actual side effects, an adverse drug reaction module 104 may be configured to learn from this new added information and more accurately predict side effects over time (e.g., for subsequent medication candidates, or the like).

An adverse drug reaction module 104 may process information about a medication candidate using one or more machine learning models to predict whether or not the medication candidate may cause each of a plurality of categories of side effects. In one embodiment (e.g., for certain medication candidates, or the like) an adverse drug reaction module 104 may process only the drug compound or other chemical structure information using a machine learning model to predict one or more side effects. In a further embodiment, an adverse drug reaction module 104 may process both chemical structure information and relationship information for other side effects (e.g., other than the one being predicted), for all side effects, or the like using one or more machine learning models to predict one or more side effects. An adverse drug reaction module 104, in some embodiments, may process chemical structure information, side effect relationship information, target information, and indication information for a medication candidate using one or more machine learning models to predict one or more side effects.

In one embodiment, chemical structure information may comprise a medication candidate's isomeric simplified molecular input line entry system (SMILES) representation and/or another computer readable indicator of the medication candidate's molecular structure. An adverse drug reaction module 104, in some embodiments, may featurize chemical structure information (e.g., a SMILES string, or the like) by extracting a computer readable set of features (e.g., a numerical vector, a vector of 1024 numbers, a vector of 2048 numbers, or the like). For example, an adverse drug reaction module 104 may featurize chemical structure information using extended connectivity fingerprints (ECFPs) (e.g., circular fingerprints), GraphConv featurization, or the like.

An adverse drug reaction module 104, in some embodiments, uses one or more machine learning models to classify “yes” or “no” for each of a plurality of side effects, side effect types, or the like. For example, in one embodiment, an adverse drug reaction module 104 may comprise different machine learning models for each of a plurality of types of side effects (e.g., 27 independent machine learning models for 27 types of side effects, or the like). The different machine learning models may comprise different types of machine learning models, such as one or more deep learning models, multitask classifiers (e.g., a robust multitask classifier, or the like), graph convolutional neural networks (e.g., for multi-label classification or the like), knowledge graph (KG) embedding models (e.g., with nodes for chemical structure information, side effect relationship information, target information, indication information, multiple potential/predicted side effects, or the like), link completion models, K nearest neighbors models, random forest models, or the like.

In certain embodiments, an adverse drug reaction module 104 may use side effect relationship information (e.g., other known side effects, correlations between different side effect types, or the like) to predict one or more other side effects for a medication candidate. In some embodiments, an adverse drug reaction module 104 comprises one or more multi-label classifier machine learning models configured to predict multiple side effects at once.

An adverse drug reaction module 104, in one embodiment, uses a machine learning model comprising a knowledge graph, to learn and/or predict relationships between entities (e.g., with nodes for chemical structure information, side effects, side effect relationship information, target information, indication information, a user's medical history, or the like). For example, an adverse drug reaction module 104 may predict one or more missing nodes, missing connections, or the like to complete a knowledge graph and/or otherwise predict one or more side effects of a medication candidate. Examples of machine learning knowledge graphs include TransE, RotatE, ComplEx, or the like.

In certain embodiments, an adverse drug reaction module 104 dynamically uses whatever information is available for a medication candidate, to predict one or more side effects. For example, an adverse drug reaction module 104 may process and use only chemical structure information, if that is the only information available, to predict one or more side effects, may process and use both chemical structure information and side effect relationship information, may process and use target information and indication information, and/or may process and use all of the above or other sub-combinations of the above depending on what information is available, to predict side effects of a medication candidate.

In some embodiments, an adverse drug reaction module 104 may perform additional processing and make new predictions as additional information becomes available, continuously improving the predictions with additional information. For example, if only chemical structure information is available, an adverse drug reaction module 104 may use a multi-task classifier, a graph convolutional network, or both and/or may leverage a knowledge graph that has other information that is available at manufacture time (e.g., target information, indication information, side effect relationship information, or the like). As more information is known, such as new uses of the medication candidate (e.g., new indication information) and/or observed side effects, or the like, an adverse drug reaction module 104 may use the additional information to update the knowledge graph, and classifiers, and/or other machine learning models to improve future predictions for this medication candidate and other new drugs.

In embodiments where an adverse drug reaction module 104 processes information using multiple machine learning models, the adverse drug reaction module 104 may combine predictions and/or other results from the multiple machine learning models to generate a list of predicted side effects. In one embodiment, an adverse drug reaction module 104 only includes a side effect in a list of predicted side effects in response to each of a plurality of machine learning models predicting the side effect (e.g., agreeing). In other embodiments, an adverse drug reaction module 104 may include a side effect in a list of predicted side effects in response to a majority of machine learning models predicting the side effect, in response to any one or more machine learning model predicting the side effect, in a round robin manner, in a voting manner, and/or in any other predefined manner. For example, in a further embodiment, an adverse drug reaction module 104 may comprise a different machine learning model for different side effects, different types or categories of side effects, or the like (e.g., different machine learning models may predict different side effects and the adverse drug reaction module 104 may combine predictions from different machine learning models into a single list, or the like).

An adverse drug reaction module 104 may provide a predicted list of side effects to doctors, pharmacists, patients, and/or other users, giving them additional information that can be used to improve drug safety. An adverse drug reaction module 104 may also be used at hospitals and/or other medical facilities to provide information about potential causes of observed conditions in patients, and whether those may be related to medications a patient is taking. An adverse drug reaction module 104, in some embodiments, may help remote and/or rural communities which may not have access to experienced doctors and/or nurses. In a further embodiment, an adverse drug reaction module 104 may be used in locations around the world to predict side effects and/or other drug reaction predictors for their local population, which may not otherwise have research done specifically for their population, ethnic groups, or the like, helping local doctors to give targeted care to their patients.

An adverse drug reaction module 104 may receive information from a researcher, from a user, from a medical professional evaluating a user, from a computing device 102, from a backend server device 108, over a data network 106, from one or more user interface elements of a hardware computing device 102 and/or a backend server device 108, or the like. An adverse drug reaction module 104 may provide a list of multiple predicted side effects for a medication candidate, a selected medication for a user based on generated lists of side effects for multiple medications, a recommendation to send a medication candidate back to a testing stage, a change in a target patient demographic for a medication candidate, to a user (e.g., a researcher, a patient, a medical professional, or the like), in a user interface of an electronic display screen of a hardware computing device 102, through an application programming interface (API), or the like.

An adverse drug reaction module 104 may receive information for a medication candidate using any appropriate techniques. For example, a frontend adverse drug reaction module 104a may be installed as an application or “app” on a hardware computing device 102 that uses a REST (representational state transfer) API call to transmit the information over the internet, a mobile telephone network, or other data network 106 to a backend adverse drug reaction module 104b installed on a backend server device 108. In another example, a researcher and/or a medical professional may have a hardware computing device 102 that is used to record information for a medication candidate and transmit the information to an adverse drug reaction module 104. In some embodiments, an adverse drug reaction module 104 may be installed on a local hardware computing device 102 such that it is not necessary to transmit the data over a data network 106.

To train a machine learning model for predicting side effects for a medication candidate, a corpus of training data may be collected. The training corpus may include examples of data where side effects of a medication are known (e.g., for existing medications). An adverse drug reaction module 104 may use a training corpus that includes multiple types of information (e.g., chemical structure information, side effect relationship information, target information, indication information, medical history information, or the like) for training a machine learning model for predicting side effects of a medication candidate.

In one embodiment, the system 100 includes one or more hardware computing devices 102. The hardware computing devices 102 and/or the one or more backend server devices 108 (e.g., computing devices, information handling devices, or the like) may include one or more of a desktop computer, a laptop computer, a mobile device, a tablet computer, a smart phone, a smart watch, a fitness band, an optical head-mounted display (e.g., a virtual reality headset, smart glasses, or the like), an HDMI or other electronic display dongle, a personal digital assistant, and/or another computing device comprising a processor (e.g., a central processing unit (CPU), a processor core, a field programmable gate array (FPGA) or other programmable logic, an application specific integrated circuit (ASIC), a controller, a microcontroller, and/or another semiconductor integrated circuit device), a volatile memory, and/or a non-volatile storage medium. In certain embodiments, the hardware computing devices 102 are in communication with one or more backend server devices 108 via a data network 106, described below. The hardware computing devices 102, in a further embodiment, are capable of executing various programs, program code, applications, instructions, functions, or the like.

In various embodiments, an adverse drug reaction module 104 may be embodied as hardware, software, or some combination of hardware and software. In one embodiment, an adverse drug reaction module 104 may comprise executable program code stored on a non-transitory computer readable storage medium for execution on a processor of a hardware computing device 102; a backend server device 108; or the like. For example, an adverse drug reaction module 104 may be embodied as executable program code executing on one or more of a hardware computing device 102; a backend server device 108; a combination of one or more of the foregoing; or the like. In such an embodiment, the various modules that perform the operations of an adverse drug reaction module 104, as described below, may be located on a hardware computing device 102; a backend server device 108; a combination of the two; and/or the like.

In various embodiments, an adverse drug reaction module 104 may be embodied as a hardware appliance that can be installed or deployed on a backend server device 108, on a user's hardware computing device 102 (e.g., a dongle, a protective case for a phone 102 or tablet 102 that includes one or more semiconductor integrated circuit devices within the case in communication with the phone 102 or tablet 102 wirelessly and/or over a data port such as USB or a proprietary communications port, or another peripheral device), or elsewhere on the data network 106 and/or collocated with a user's hardware computing device 102. In certain embodiments, an adverse drug reaction module 104 may comprise a hardware device such as a secure hardware dongle or other hardware appliance device (e.g., a set-top box, a network appliance, or the like) that attaches to another hardware computing device 102, such as a laptop computer, a server, a tablet computer, a smart phone, or the like, either by a wired connection (e.g., a USB connection) or a wireless connection (e.g., Bluetooth®, Wi-Fi®, near-field communication (NFC), or the like); that attaches to an electronic display device (e.g., a television or monitor using an HDMI port, a DisplayPort port, a Mini DisplayPort port, VGA port, DVI port, or the like); that operates substantially independently on a data network 106; or the like. A hardware appliance of an adverse drug reaction module 104 may comprise a power interface, a wired and/or wireless network interface, a graphical interface (e.g., a graphics card and/or GPU with one or more display ports) that outputs to a display device, and/or a semiconductor integrated circuit device as described below, configured to perform the functions described herein with regard to an adverse drug reaction module 104.

An adverse drug reaction module 104, in such an embodiment, may comprise a semiconductor integrated circuit device (e.g., one or more chips, die, or other discrete logic hardware), or the like, such as a field-programmable gate array (FPGA) or other programmable logic, firmware for an FPGA or other programmable logic, microcode for execution on a microcontroller, an application-specific integrated circuit (ASIC), a processor, a processor core, or the like. In one embodiment, an adverse drug reaction module 104 may be mounted on a printed circuit board with one or more electrical lines or connections (e.g., to volatile memory, a non-volatile storage medium, a network interface, a peripheral device, a graphical/display interface. The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of an adverse drug reaction module 104.

The semiconductor integrated circuit device or other hardware appliance of an adverse drug reaction module 104, in certain embodiments, comprises and/or is communicatively coupled to one or more volatile memory media, which may include but is not limited to: random access memory (RAM), dynamic RAM (DRAM), cache, or the like. In one embodiment, the semiconductor integrated circuit device or other hardware appliance of an adverse drug reaction module 104 comprises and/or is communicatively coupled to one or more non-volatile memory media, which may include but is not limited to: NAND flash memory, NOR flash memory, nano random access memory (nano RAM or NRAM), nanocrystal wire-based memory, silicon-oxide based sub-10 nanometer process memory, graphene memory, Silicon-Oxide-Nitride-Oxide-Silicon (SONOS), resistive RAM (RRAM), programmable metallization cell (PMC), conductive-bridging RAM (CBRAM), magneto-resistive RAM (MRAM), dynamic RAM (DRAM), phase change RAM (PRAM or PCM), magnetic storage media (e.g., hard disk, tape), optical storage media, or the like.

The data network 106, in one embodiment, includes a digital communication network that transmits digital communications. The data network 106 may include a wireless network, such as a wireless cellular network, a local wireless network, such as a Wi-Fi network, a Bluetooth® network, a near-field communication (NFC) network, an ad hoc network, and/or the like. The data network 106 may include a wide area network (WAN), a storage area network (SAN), a local area network (LAN), an optical fiber network, the internet, or other digital communication network. The data network 106 may include two or more networks. The data network 106 may include one or more servers, routers, switches, and/or other networking equipment. The data network 106 may also include one or more computer readable storage media, such as a hard disk drive, an optical drive, non-volatile memory, RAM, or the like.

The one or more backend server devices 108, in one embodiment, may include one or more network accessible computing systems such as one or more web servers hosting one or more web sites, an enterprise intranet system, an application server, an API server, an authentication server, or the like. A backend server device 108 may include one or more servers located remotely from the hardware computing devices 102. A backend server device 108 may include at least a portion of the adverse drug reaction modules 104, may comprise hardware of an adverse drug reaction module 104, may store executable program code of an adverse drug reaction module 104 in one or more non-transitory computer readable storage media, and/or may otherwise perform one or more of the various operations of an adverse drug reaction module 104 described herein.

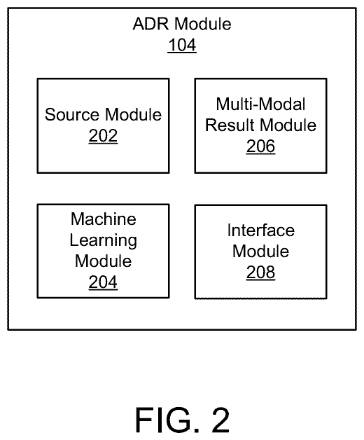

FIG. 2 depicts one embodiment of an adverse drug reaction module 104. The adverse drug reaction module 104, in certain embodiments, may be substantially similar to one or more of a device adverse drug reaction module 104a and/or a backend adverse drug reaction module 104b, as described above with regard to FIG. 1. The adverse drug reaction module 104, in the depicted embodiment, includes a source module 202, a machine learning module 204, a multi-modal result module 206, and an interface module 208.

In one embodiment, the source module 202 is configured to receive multiple types of information for a medication candidate. For example, in various embodiments, a source module 202 may receive chemical structure information, side effect relationship information, target information, indication information, a medical history and/or other medical records for a user, and/or other types of data relevant to a side effect of a medication candidate.

In certain embodiments, a plurality of source modules 202 disposed on a plurality of different computing devices 102 may receive data for a plurality of different users (e.g., medical histories and/or other medical records, detecting side effects, or the like). For example, a plurality of distributed source modules 202 may collect data samples for a medical trial, to train a machine learning model for predicting side effects, or the like.

The source module 202, in one embodiment, may store received information on a computer readable storage medium of a computing device 102, 110, so that the machine learning module 204 may access and/or process the received information to predict side effects, train machine learning models for predicting side effects, or the like; so that the interface module 208 may provide the received information to one or more authorized users; and/or so that the received information is otherwise accessible for use. In another embodiment, the source module 202 may provide received information directly to the machine learning module 204 for training and/or for predicting side effects (e.g., without otherwise storing the data, temporarily storing and/or caching the data, or the like). The source module 202 may store and/or organize received information in a database and/or other predefined data structure accessible by the machine learning module 204, the multi-modal result module 206, the interface module 208, or the like.

By storing information for a medication candidate, in certain embodiments, the source module 202 may enable the machine learning module 204 and/or the multi-modal result module 206 to dynamically predict side effects for one or more medication candidates. For example, the source module 202 may store information for one or more medication candidates on a hardware computing device 102, on a backend server device 108 in communication with a hardware computing device 102 over a data network 106, or the like, enabling the machine learning module 204 to predict side effects.

In one embodiment, a machine learning module 204 is configured to process information from a source module 202 using one or more machine learning models to predict a list of side effects for a medication candidate. For example, in some embodiments, a machine learning module 204 may use different machine learning models to process different types of information (e.g., chemical structure information, side effect relationship information, target information, indication information, medical history information, or the like).

In one embodiment a multi-modal result module 206 is configured to predict a list of multiple side effects for a medication candidate based on multiple types of information from a source module 202 and/or multiple predictions from a machine learning module 204, or the like. For example, the multi-modal result module 206 may combine or otherwise process and/or analyze predictions for multiple types of information for a medication candidate, into a single list of side effects or other result indicating whether or not each side effect is likely for a medication candidate.

The multi-modal result module 206 may use one or more rules to determine a list of side effects and/or other result based on multiple predictions, multiple types of information, or the like. In one embodiment, the multi-modal result module 206 may use a conservative rule and may be configured to predict a side effect in response to all of multiple predictions indicating the side effect is likely for a medication candidate.

In a further embodiment, the multi-modal result module 206 may use a voting rule and may be configured to predict a side effect for a medication candidate in response to a majority of multiple predictions indicating the side effect is likely (e.g., at least two out of three, three out of four or five, four out of six or seven, five out of eight or nine, six out of ten or eleven, or the like).

In one embodiment, the multi-modal result module 206 may use an aggressive rule, and may be configured to predict a side effect is likely for a medication candidate in response to at least one of multiple predictions indicating that the side effect is likely for a medication candidate (e.g., if any one type of information, any one machine learning model, or the like indicated that the side effect is likely for the medication candidate, the multi-modal result module 206 predicts that the side effect is likely for the medication candidate).

In certain embodiments, the multi-modal result module 206 may use an override rule allowing a prediction based on one type of information to override one or more other predictions, types of information, or the like. For example, the multi-modal result module 206 may be configured to determine that a side effect is likely for a medication candidate, if a predefined machine learning model indicates that it is likely, if two predefined machine learning models indicate that it is likely, or the like. In one embodiment, the multi-modal result module 206 may be configured to determine that a side effect is likely for a medication candidate in response to either a researcher or other user indicating the side effect is likely (e.g., an override) or a machine learning model determining the side effect is likely.

The interface module 208, in certain embodiments, is configured to execute on a hardware computing device 102 (e.g., of a user such as a researcher investigating a medication candidate, a user, or the like) and/or on a backend server device 108, or the like. In one embodiment, the interface module 208 may be configured to provide a user interface to a researcher, a medical professional, a patient, and/or to another user. In a further embodiment, the interface module 208 is configured to provide an API to the source module 202, the machine learning module 204, the multi-modal result module 206, other interface modules 208, other adverse drug reaction modules 104, hardware computing devices 102, backend server devices 108, or the like.

The interface module 208, in one embodiment, is configured to cooperate with the source module 202, the machine learning module 204, and/or the multi-modal result module 206. For example, the source module 202 may be configured to receive multiple types of information for a medication candidate through a user interface of the interface module 208 displayed on an electronic display screen of a hardware computing device 102 and to provide the multiple types of information to the machine learning module 204 using an API of the interface module 208, or the like. In a further example, the interface module 208 may be configured to receive a list of side effects or other result for a medication candidate from the multi-modal result module 206 over an API of the interface module 208, and the interface module 208 may be configured to display the single result in a user interface on an electronic display screen of a hardware computing device 102.

In one embodiment, the interface module 208 provides one or more users with access to received types of information from the source module 202 (e.g., chemical structure information, side effect relationship information, target information, indication information, medical history information, or the like), to lists of predicted side effects and/or other results from the machine learning module 204 and/or the multi-modal result module 206, or the like. The interface module 208 may allow a user to access received types of information, lists of predicted side effects and/or other results, or the like from multiple locations (e.g., from a mobile app on a mobile computing device 102, from a web browser of a different computing device 102 accessing a web server of a backend server device 108, or the like).

In certain embodiments, the interface module 208 may enforce access control permissions (e.g., for privacy, for security, for HIPAA compliance, or the like) by authenticating users (e.g., with a username and password or other authentication credentials) and providing the users access to types of information, lists of predicted side effects or other results, or the like based on access control permissions associated with the user.

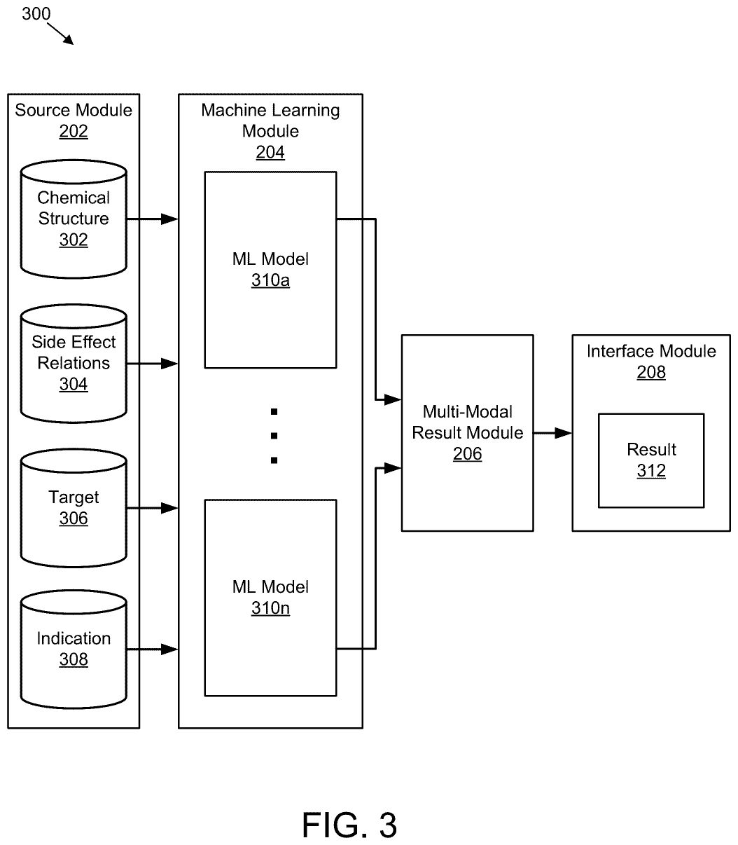

FIG. 3 depicts one embodiment of a system 300 for machine learning adverse drug reaction prediction. In the depicted embodiment, the source module 202 receives chemical structure information 302, side effect relationship information 304, target information 306, and/or indication information 308 and provides the received types of information 302, 304, 306, 308 to the machine learning module 204. The machine learning module 204 processes the received information 302, 304, 306, 308 using one or more machine learning models 310a-n to predict one or more side effects for a medication candidate.

In the depicted embodiment, the multi-modal result module 206 analyzes and/or combines the predicted side effects from the one or more machine learning models 310a-n to determine a list 312 of predicted side effects for a medication candidate (e.g., using a conservative rule, a voting rule, an aggressive rule, an override rule, a decision tree, or the like). The multi-modal result module 206 provides the list 312 to the interface module 208. The interface module 208 may display the list 312 to a user (e.g., a researcher, a medical professional, a patient, and/or another user) on an electronic display screen of a hardware computing device 102, may provide the result 314 over an API in response to an API request, or the like.

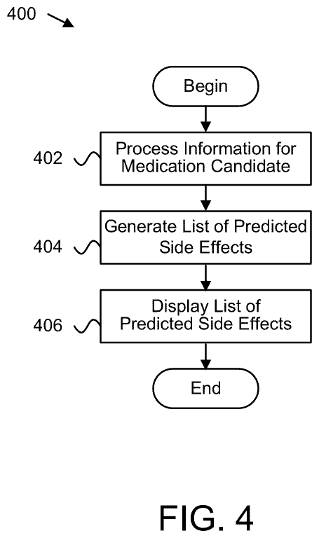

FIG. 4 depicts one embodiment of a method 400 for machine learning adverse drug reaction prediction. The method 400 begins, and an adverse drug reaction module 104 processes 402 one or more of chemical structure information, side effect relationship information, target information, and indication information for a medication candidate using one or more machine learning models. An adverse drug reaction module 104 generates 404 a list of multiple predicted side effects for a medication candidate based on the processing 402. An adverse drug reaction module 104 displays 406, to a user, the generated 404 list of multiple predicted side effects for a medication candidate on an electronic display screen for a hardware computing device 102 and the method 400 ends.

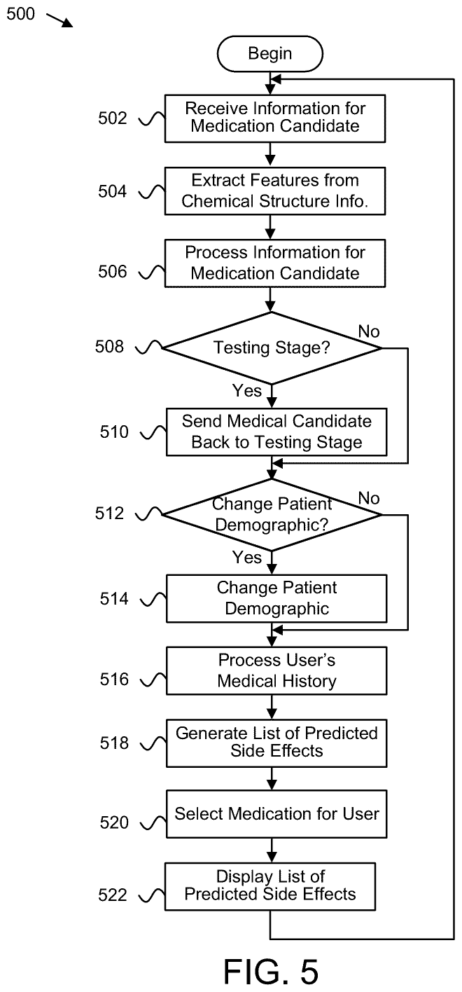

FIG. 5 depicts one embodiment of a method 500 for machine learning adverse drug reaction prediction. The method 500 begins, and an adverse drug reaction module 104 receives 502 one or more of chemical structure information, side effect relationship information, target information, indication information, and/or medical history information for a medication candidate. An adverse drug reaction module 104 extracts 504 a plurality of features from at least the chemical structure information.

An adverse drug reaction module 104 processes 506 the received 502 information and/or the extracted 504 features using one or more machine learning models. An adverse drug reaction module 104 determines 508, based on the processing 506, whether or not to send a medication candidate back to a testing stage. If an adverse drug reaction module 104 determines 508 to send a medication candidate back to a testing stage, the adverse drug reaction module 104 sends 510 the medication candidate back to the testing stage.

An adverse drug reaction module 104 determines 512, based on the processing 506, whether or not to change a patient demographic for a medication candidate. If an adverse drug reaction module 104 determines 512 to change a patient demographic for a medication candidate, the adverse drug reaction module 104 changes 514 the patient demographic for the medication candidate.

An adverse drug reaction module 104 processes 516 a user's medical history using one or more machine learning models. An adverse drug reaction module 104 generates 518 a list of multiple predicted side effects for a medication candidate based on the processing 506, 516. An adverse drug reaction module 104 generates 518 a list of multiple predicted side effects for a medication candidate based on the processing 506, 516.

An adverse drug reaction module 104 selects 520 a medication for a suer from multiple medications (e.g., including a medication candidate) based on the generated 518 list of multiple predicted side effects for the medication candidate and on other generated lists of multiple predicted side effects for the multiple medications. An adverse drug reaction module 104 displays 522, to a user, the generated 518 list of multiple predicted side effects, the determination 508 to send 510 the medication candidate back to a testing stage, the determination 512 to change 514 a patient demographic for the medication candidate, the selected 520 medication for a user, and/or other information and the method 500 continues (e.g., for subsequent medication candidates, or the like).

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims, or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules (or components), in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flowchart diagrams and/or schematic block diagrams in the Figures illustrate the architecture, functionality, and operation of possible embodiments of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative embodiments, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An apparatus, comprising:
a processor; and
a memory, the memory storing computer program code executable by the processor to perform operations, the operations comprising:
training one or more machine learning models using a corpus of training data, the corpus of training data comprising one or more of medication data, chemical structure information, side effect information, target information, indication information, and medical history information;
processing one or more of chemical structure information, side effect relationship information, target information, and indication information for a medication candidate using the one or more machine learning models, wherein different machine learning models of the one or more machine learning models are independently used to separately predict each of a plurality of side effects for the medication candidate;
generating a list of multiple predicted side effects for the medication candidate based on the side effects that the machine learning models predict, the list generated according to one or more rules that define whether a predicted side effect is included in the list based on whether a combination of the one or more machine learning models that predict the side effect satisfies a threshold number associated with the one or more rules; and
displaying, to a user, the generated list of multiple predicted side effects for the medication candidate on an electronic display screen for a hardware computing device.

2. The apparatus of claim 1, the operations further comprising processing a user's medical history using the one or more machine learning models, wherein the generated list of multiple predicted side effects is based at least partially on the user's medical history.

3. The apparatus of claim 2, the operations further comprising selecting a medication for the user from multiple medications including the medication candidate based on the generated list of multiple predicted side effects for the medication candidate and other generated lists of multiple predicted side effects for the multiple medications.

4. The apparatus of claim 1, wherein the one or more machine learning models comprise at least a first machine learning model and a second machine learning model and the first machine learning model and the second machine learning model each predict whether the medication candidate has each of a plurality of predefined possible side effects.

5. The apparatus of claim 4, wherein each specific side effect of the multiple predicted side effects for the medication candidate is included in the generated list in response to at least one of the first and second machine learning models predicting that the medication candidate has the specific side effect.

6. The apparatus of claim 1, the operations further comprising sending the medication candidate back to a testing stage in response to the generated list of multiple predicted side effects for the medication candidate.

7. The apparatus of claim 1, the operations further comprising changing a patient demographic for the medication candidate based on the generated list of multiple predicted side effects for the medication candidate.

8. The apparatus of claim 1, the operations further comprising extracting a plurality of features from the chemical structure information, the one or more machine learning models processing the extracted plurality of features from the chemical structure information.

9. The apparatus of claim 1, wherein the target information for the medication candidate comprises a protein target of the medication candidate.

10. The apparatus of claim 1, wherein the one or more machine learning models comprise a knowledge graph with nodes for the chemical structure information, the side effect relationship information, the target information, the indication information, and the multiple predicted side effects.

11. The apparatus of claim 1, wherein the one or more machine learning models comprise a K nearest neighbors model.

12. The apparatus of claim 1, wherein the one or more machine learning models comprise a random forest model.

13. The apparatus of claim 1, wherein the one or more machine learning models comprise one or more of a multitask classifier, a robust multitask classifier, and a graph convolutional neural network.

14. A computer program product comprising a non-transitory computer readable storage medium storing computer program code executable to perform operations, the operations comprising:
training one or more machine learning models using a corpus of training data, the corpus of training data comprising one or more of medication data, chemical structure information, side effect information, target information, indication information, and medical history information;
processing one or more of chemical structure information, side effect relationship information, target information, and indication information for a medication candidate using the one or more machine learning models, wherein different machine learning models of the one or more machine learning models are independently used to separately predict side effects for the medication candidate;
generating a list of multiple predicted side effects for the medication candidate based on the side effects that the machine learning models predict, the list generated according to one or more rules that define whether a predicted side effect is included in the list based on whether a combination of the one or more machine learning models that predict the side effect satisfies a threshold number associated with the one or more rules; and
displaying, to a user, the generated list of multiple predicted side effects for the medication candidate on an electronic display screen for a hardware computing device.

15. The computer program product of claim 14, the operations further comprising processing a user's medical history using the one or more machine learning models, wherein the generated list of multiple predicted side effects is based at least partially on the user's medical history.

16. The computer program product of claim 15, the operations further comprising selecting a medication for the user from multiple medications including the medication candidate based on the generated list of multiple predicted side effects for the medication candidate and other generated lists of multiple predicted side effects for the multiple medications.

17. The computer program product of claim 14, wherein the one or more machine learning models comprise at least a first machine learning model and a second machine learning model, the first machine learning model and the second machine learning model each predict whether the medication candidate has each of a plurality of predefined possible side effects, and each specific side effect of the multiple predicted side effects for the medication candidate is included in the generated list in response to at least one of the first and second machine learning models predicting that the medication candidate has the specific side effect.

18. The computer program product of claim 14, the operations further comprising sending the medication candidate back to a testing stage in response to the generated list of multiple predicted side effects for the medication candidate.

19. The computer program product of claim 14, the operations further comprising changing a patient demographic for the medication candidate based on the generated list of multiple predicted side effects for the medication candidate.

20. A method comprising:
training one or more machine learning models using a corpus of training data, the corpus of training data comprising one or more of medication data, chemical structure information, side effect information, target information, indication information, and medical history information;
processing one or more of chemical structure information, side effect relationship information, target information, and indication information for a medication candidate using the one or more machine learning models, wherein different machine learning models of the one or more machine learning models are independently used to separately predict side effects for the medication candidate;
generating a list of multiple predicted side effects for the medication candidate based on the side effects that the machine learning models predict, the list generated according to one or more rules that define whether a predicted side effect is included in the list based on whether a combination of the one or more machine learning models that predict the side effect satisfies a threshold number associated with the one or more rules; and
displaying, to a user, the generated list of multiple predicted side effects for the medication candidate on an electronic display screen for a hardware computing device.
